(19) United States
(12) Patent Application Publication
(10) Pub. No.: US 2018/0185188 A1
(43) Pub. Date: Jul. 5, 2018

(54) INTRAORAL MANDIBULAR ADVANCEMENT DEVICE FOR TREATMENT OF SLEEP DISORDERS

(71) Applicant: Steven B. Lamberg, Northport, NY (US)

(72) Inventor: Steven B. Lamberg, Northport, NY (US)

(21) Appl. No.: 15/397,571

(22) Filed: Jan. 3, 2017

(51) Int. Cl. A61F 5/56

(52) U.S. Cl. CPC A61F 5/566; A61F 2005/563

(57) ABSTRACT

An intraoral mandibular advancement device for treatment of sleep disorders includes a maxillary appliance having a protrusive element distending from the anterior portion of the maxillary appliance, and a mandibular appliance having a polymeric shell with recesses to receive and removably engage the mandibular teeth. A lingual spacer extends posteriorly from the anterior portion of the mandibular appliance to contact the anterior aspect of the protrusive element and thereby cause mandibular advancement. An anterior stop extends upward from the occlusal surface of the anterior portion of the mandibular appliance to contact the anterior portion of the maxillary appliance and thereby maintain separation between the anterior teeth and provide a mechanism, by way of its vertical reduction, to reinstate posterior contacts as the jaw moves forward. Posterior stops on the occlusal surface of the posterior portions of either appliance maintain separation between the posterior teeth.

### BACKGROUND OF THE INVENTION

### Field of the Invention

The present invention relates generally to the field of devices for treatment of sleep disorders. More specifically, the present invention discloses an intraoral mandibular advancement device for the treatment of snoring, obstructive sleep apnea, gastroesophageal reflux disease, and bruxism that prevents overloading of the tempromandibular joint (TMJ) by employing posterior supports and an adjustable anterior stop.

### Statement of the Problem

Among the health problems associated with sleep disorders are inter alia, snoring, gastroesophageal reflux disease (“GERD”), obstructive sleep apnea (“OSA”), bruxism, diabetes, high blood pressure (hypertension), and increased risk of stroke or heart attack. The most widespread sleep disorder is snoring which is a common affliction that affects tens of millions of people worldwide, and can potentially have very serious health and social consequences. Snoring is a sound produced by the vibration of tissue caused by a breathing obstruction during sleep. There are many factors associated with snoring including, but not limited to: heredity, body weight, age, gender, smoking history, general pharyngeal and nasal anatomy, tonsils, or soft palate deformities, use of alcohol, allergies, and sleep position. Commonly, both the snorer and the snorer's sleep partner lose sleep due to the snorer's snoring. The sleep partner is awakened by the sounds caused by snoring, perhaps repeatedly each night. Movement by the sleep partner in subsequently attempting to fall back asleep may then awaken the snorer, if the noise of his or her own snoring has not already done so. Lack of sleep subsequently leads to daytime fatigue, a compromised immune system, poor mental and emotional health, irritability and lack of productivity, which can lead to further repercussions. Snoring is thus problematic for many people.

Obstructive sleep apnea is a potentially lethal sleep and breathing disorder defined as the cessation of breathing for 10 seconds or more (an apnea) at least five times per hour of sleep. In severe cases, individuals can wake up 300 times per night. When breathing is interrupted by an obstruction in the airway, the body reacts by waking enough to start breathing again. Arousals may occur hundreds of times each night, and may not fully awaken the individual, who remains otherwise unaware of the loud snoring, choking and gasping for air typically associated with OSA.

Although not all snorers have OSA, snoring is a cardinal symptom of OSA and clearly indicative of and associated therewith. Many OSA sufferers do not receive a sufficient amount of sleep due to repeated apneatic events and arousals which act to prevent REM and deep stage sleep, which can lead to chronic daytime exhaustion and long-term cardiovascular stress. OSA is the direct cause of at least 38,000 deaths each year. An estimated 30 million Americans suffer from some degree of OSA, yet less than 3% are currently undergoing any type of treatment.

Pathogenesis of OSA involves a combination of reduced upper airway size and altered upper airway muscle activity, which causes oral tissue to collapse, and hence a blockage to occur. Other factors which are thought to contribute to OSA include tongue size, soft palate volume, a retrognathic mandible, an anteroposterior discrepancy between the maxilla and the mandible, and obesity.

Snoring and OSA are often associated, as generally both are caused by blockage of the pharyngeal airway by, for example, excess tissue when various muscles of the body, including the tongue, relax. As the tongue relaxes, it moves posteriorly, blocking the breathing airway. When the breathing airway is blocked, exhaled air is forced through the airway with increased velocity thereby causing vibration of the tongue, tissue, or other obstruction, thereby creating noise. Snoring is caused by the partial obstruction of breathing during sleep while OSA occurs when the tongue and soft palate collapse onto the back of the throat and completely block the pharyngeal airway, thereby stopping breathing during sleep and restricting the flow of essential oxygen. Thus, a correlation between snoring and OSA is generally recognized in the medical community.

Many attempts have been made to reduce or eliminate snoring and OSA in individuals. Various types of surgery, including tracheostomy, surgery of the soft palate and oropharynx, and reconstructive surgery have been utilized in the treatment of snoring and OSA. Invasive surgery however is costly and not without risk, which effectively eliminates this modality as a truly viable solution to snoring and OSA for the general population. Indeed, many practitioners and patients alike would generally seek to avoid surgical intervention and would welcome a minimally invasive route—a general object and feature of the instant invention.

It has been recognized that opening or enlarging an airway that may be constricted due to collapse of tissue about the oropharynx will normalize the airflow to the lungs and in doing so will have a direct effect of resolving snoring and OSA as well as diminishing pressure fluctuations in the esophagus which have been associated with GERD. The prior art includes devices that are designed to restrict the velocity of the ingress and egress of air with the goal of reducing or eliminating snoring. It is scientifically concluded herein that by opening the aperture of the airway and maintaining the same, the consequences of a collapsed airway are avoided. When the airway is collapsed, the velocity of air flow increases and causes the vibration of the tongue, soft palate, and other tissue present in the pharyngeal wall during sleep. Thus, when expanded and maintained by the invention herein, the airflow is also maintained and snoring and OSA (where it theretofore resulted therefrom) are minimized or eliminated.

For example, U.S. Pat. No. 6,263,877 to Gall shows a device which essentially consists of a mouth guard with a small aperture for which are can pass through. The amount of air flow is therefore directly proportional to the size of the aperture. U.S. Pat. No. 5,642,738 to Lilly shows a device which is designed to eliminate snoring via a membrane which is placed on the exterior surface of the anterior teeth to reduce the volume of air which is allowed to flow in and out of the mouth. U.S. Pat. No. 4,817,636 to Woods likewise shows a device which seeks to eliminate snoring via a membrane which is placed on the exterior surface of the anterior teeth to reduce the volume of air which is allowed to flow in and out of the mouth. Yet, reduction of airflow can cause a cascade of other symptomology and situations, including potential OSA.

Also known in the art are devices which seek to reduce or eliminate snoring or OSA by inhibiting vibration of the soft palate. For example, U.S. Pat. No. 6,467,485 to Schmidt shows a device made of a flexible material which is placed over the soft palate in an attempt to inhibit vibration of the soft palate. U.S. Pat. No. 4,669,459 to Spiewak shows an intraoral device which seeks to eliminate snoring by positioning a button on the soft palate to prevent vibration thereof. Yet, such devices do not address the source of the problem or intervene to increase the airway (as does the instant invention), and thus the cascade effect is still a present risk if such devices are employed.

Various devices have sought to alleviate snoring by aiming to keep the pharyngeal airway open to prevent the collapse of the tongue and soft tissues in the back of the throat, yet are bulky and uncomfortable, often resulting in reduced patient compliance. For example, U.S. Pat. No. 6,386,201 to Fard shows a pillow which seeks to keep the user's head in proper position to make snoring less likely. U.S. Pat. No. 4,366,815 to Broomes and U.S. Pat. No. 5,357,981 encompass other designs aimed at maintaining the user's body in a position which makes snoring less likely. U.S. Pat. No. 6,668,834 shows a device which seeks to eliminate or reduce snoring by maintaining the user's chin above the user's chest while in the supine position in order to facilitate breathing as well as to aid in the prevention of snoring.

Positioning of the body does not eliminate the cause of snoring. Since tongue posture appears to have a substantial effect on upper airway morphology, several devices show a design which seeks to hold the tongue in a forward position in order to increase the diameter of the pharyngeal airway during sleep. For example, U.S. Pat. No. 6,494,209 to Kulick shows a device which consists essentially of a mouthpiece with an anterior suction mechanism which creates a vacuum which acts to maintain the tongue in an anterior position. U.S. Pat. No. 6,408,851 shows a device which clamps the tongue in order to maintain the tongue in a forward position. U.S. Pat. No. 6,055,986 to Meade shows a device for the reduction of snoring which encompasses both upper and lower positions which together form a cavity which encompasses the user's tongue when in use to maintain the tongue in a predetermined position or to prevent the tongue from moving posteriorly and thus blocking the pharyngeal airway. U.S. Pat. No. 5,988,170 shows a snoring prevention apparatus consisting essentially of a mask having a tongue depressing member, which acts to maintain the tongue in an anterior position during use. U.S. Pat. No. 6,845,774 to Gaskell shows a mandibular splint which acts to maintain the user's jaws at a predetermined space apart to prevent the tongue from moving posteriorly in order to open the breathing passage. One can only image how truly uncomfortable such devices must be if used at all, and thus the likelihood of patient compliance is small to nil.

There is also recognition of those of ordinary skill in the art of a correlation between OSA and nocturnal gastroesophageal reflex disease (“GERD”). Both OSA and GERD involve a reduction in air pressure in the trachea and esophagus caused by blockage during sleeping, thereby resulting in an increase in the draw of pressure through the esophagus as the diaphragm distends. While the body tends to increase breathing by such distention, the blockage results in the pulling of fluid upwardly in the esophagus and hence results in GERD for the snorer or OSA patient. Yet, the currently recommended solutions involve some version of a “CPAP” (continuous positive airway pressure) or pharmacological intervention. The former involves a face mask and a pump to provide pressure directly to the trachea of the patient which is cumbersome and unaesthetic. The latter obviously includes a plethora of side effects associated with the pharmacology employed, while not directly addressing the physical/mechanical source of the problem.

It is herein recognized that advancing the mandible in an anterior position relative to the maxilla during sleep opens the pharyngeal airway by indirectly urging the tongue forward to stimulate activity of the muscles in the tongue and thereby also increases the forward rigidity of the tongue. Since the tongue attaches to the posterior portion of the mandibular symphysis, advancing the mandible forward relative to the maxilla also pulls the tongue forward, thus preventing the tongue from obstructing the pharyngeal airway. Since the palatoglossus muscle attaches from the tongue to the soft palate, resultant forward movement of the tongue thereby creates tension on the soft palate thereby reducing vibration. Mandibular advancement devices therefore function to move the lower jaw, and hence the tongue forward to open the oropharynx. Snoring thus decreases proportionally with the increase in airway size or diameter.

It should therefore be appreciated that by so urging, and as an object of the instant invention, there can be a positive effect on reduction of snoring and OSA, and also upon GERD which is associated with the existence of large intrapleural pressure swings, which occur during apneatic events.

A number of different appliances for the treatment of snoring or OSA which move the mandible forward relative to the maxilla have been suggested, yet such devices typically fit over both the arches of teeth and are thus large and cumbersome. Several such different devices allegedly for the treatment of snoring or OSA move the mandible forward relative to the maxilla can be observed in the following U.S. Patents: Halstrom, U.S. Pat. No. 6,729,335; Thornton, U.S. Pat. No. 6,516,805; Orrico, U.S. Pat. No. 6,170,485; Belfer, U.S. Pat. No. 6,092,523; Bergersen, U.S. Pat. No. 6,129,084; Fenton, U.S. Pat. No. 5,499,633; Tomasi, U.S. Pat. No. 5,313,960; Hays, U.S. Pat. No. 5,277,202; Shapiro, U.S. Pat. No. 5,177,816; and Strong, U.S. Pat. No. 6,526,982. These devices consist essentially of acrylic or elastomeric upper and lower bite trays which fit over both the maxillary and mandibular teeth, respectively, with some connecting means to shift the user's bite so that the mandible is urged forward relative to the maxilla. Thus, to achieve anterior movement of the mandible, these devices essentially move one jaw against the other in a cumbersome manner which no doubt has an effect on patient compliance.

Such appliances which fit over both arches of teeth were derived from the orthodontic domain which it has ordinarily been considered prudent to cover all the teeth in order to prevent undesired movement (eruption) of any teeth not covered by the device resulting from a lack of opposition. Studies have shown however that partial coverage of the teeth during sleep does not result in undesired eruption of uncovered teeth, therefore rendering previous concerns unwarranted. These devices are therefore unnecessarily bulky and difficult to fit over the user's teeth. Bulky devices also occupy a large portion of the oropharyngeal volume, thus making breathing, and the passage of air around the bulky device difficult—the very antithesis of the goal herein sought: to increase the dimensional size of the airway. Breathing difficulty substantially reduces the efficacy of such devices to treat snoring or OSA, let alone the secondary effects of GERD caused by airway obstruction.

Many devices claimed for treatment of snoring or OSA which move the mandible forward relative to the maxilla fit over both the arches of teeth and also move one jaw substantially against the other, thereby posing other potentially damaging effects. Mandibular advancement devices that fit over both the maxillary and mandibular teeth are typically held nearly stationary, thereby restricting movement, causing discomfort, and potential permanent repositioning of the jaw. Since these types of devices restrict the user's natural lateral movements as well as anterior and posterior movements, continued use can potentially aggravate the tempromandibular joint (TMJ) and the related facial musculature, which would worsen over time, with continued use. Thus, it is an object of the instant invention to permit such movements, increase comfort and compliance, and additionally avoid TMJ effects.

Also shown in the art are appliances which are worn only upon the user's maxillary teeth, thus leaving the mandibular teeth uncovered. For example, U.S. Pat. No. 5,915,385 to Hakimi discloses a snore and stress relief device which seeks to engage the user's upper dentition and includes an anterior extension which appears to advance the user's lower jaw forward with respect to the user's upper jaw such that the user's upper airway is enlarged and the passage of air through the upper airway is facilitated. The device disclosed in Hakimi has a posterior position which fits against the dorsal surface of the user's soft palate to secure the device, which may also cause gagging or other discomfort, thus reducing or eliminating efficacy.

Although prior devices may have effect in treating snoring and OSA by moving the mandible forward relative to the maxilla to open the airway, such devices have unwanted side effects or are ineffective due to, inter alia, their bulk. As stated herein, many of the devices which appear to be designed to alleviate snoring and OSA restrict the user's natural nocturnal movements of the lower jaw which can potentially aggravate the tempromandibular joint and related jaw muscles and ligaments. Due to their bulk, devices prior to that shown herein that claim a design to eliminate or reduce snoring or OSA rely largely on the user's ability to breathe through the nose. Thus, such devices would not function properly with users who have difficulty breathing through their noses. Furthermore, many of these devices must be removed or unhinged or otherwise disengaged to allow the user freedom to speak, swallow, or drink water, thus creating serious inconvenience to the user (and poor aesthetics). It is thus an object of the instant invention to overcome these shortcomings in prior devices.

Gastroesophageal reflex disease (“GERD”), also known as acid reflux, is a chronic condition which affects at least 5 to 7% of the global population, including over seven million people (reportedly as high as 30 million) in the United States alone. GERD is characterized by movement of the liquid contents of the stomach from the stomach into the esophagus. Almost everyone experiences gastroesophageal reflux at some time or another. When reflux is frequent or severe enough to cause more significant problems, it is characterized as GERD. GERD can cause serious complications including inflammation of the esophagus from stomach acid that causes bleeding or ulcers. Asthma, chronic cough, and pulmonary fibrosis may be aggravated or even caused by GERD.

The liquid content of the stomach typically contains acid, pepsin, and bile, which are harmful to the delicate lining of the esophagus when regurgitated. Damage to the esophageal lining can result in scarring and narrowing of the esophagus, and can be linked to the development of esophageal cancer. Pressure created by the lower esophageal sphincter (“LES”) and diaphragm surrounding the LES ordinarily create a barrier to reflux. During snoring or an apneatic event, however, a large negative intrapleural pressure swing often occurs facilitating reflex events. Thus, blockage of the esophageal airway creates a negative pressure which allows reflux to occur by facilitating the upward movement of the liquid contents of the stomach. When in an upright position, most refluxed liquid naturally flows back into the stomach due to the force of gravity. When in a supine position however, most refluxed liquid is forced upwardly, into the esophagus, typically causing damage thereto. Due to the increased potential for reflex during sleep in the supine position, it is therefore beneficial to have a method of treatment which can be applied during sleeping hours to maintain the pressure system without creating negative esophageal pressure and concomitant potential reflex.

Symptoms of gastroesophageal reflux disease include regurgitation, heartburn, difficulty swallowing, chest pain and nausea. As a chronic disease, treatment of gastroesophageal reflux disease typically continues indefinitely. It is therefore preferable that a method of treatment for gastroesophageal reflux disease is comfortable for nightly use.

Continuous Positive Airway Pressure (“CPAP”) therapy has been shown to reduce GERD by increasing intra-esophageal pressure. Yet these devices have drawbacks, as herein stated, and thus are not of choice especially if non-invasive, easily placeable and removable, and not uncomfortable alternatives, like that of the instant invention, are available. Although there exists a broad range of dentally-retained intraoral appliances worn at night for the treatment of snoring and obstructive sleep apnea, no such appliances heretofore satisfied such criteria and, also, were directed at treatment of GERD. It is thus an object of the instant invention to present a noninvasive alternative to drug therapy and surgical intervention for the treatment of GERD.

Bruxism is the technical description for clenching, gnashing or grinding of teeth. Bruxing is common in almost all individuals and can occur subconsciously or when an individual becomes stresses or aggravated. The risk of bruxism increases with age, stress, and use of caffeine, nicotine and other drugs. In most cases, bruxism is mild and does not require treatment. When bruxing is more frequent however it can lead to jaw disorders, headaches, earaches, damaged teeth, chronic facial pains, and other problems. Therapy such as muscle relaxers and Botox are currently used as remedies for bruxism. Alleged dental solutions include mouth guards and splints which appear to be designed to prevent damage to the teeth or decrease muscle activity. For example, U.S. Pat. No. 7,004,172 to Zacco shows a device which seeks to separate a user's mandibular and maxillary teeth by creating a space in the posterior portion of said mandibular and maxillary teeth. U.S. Pat. No. 6,886,566 to Eubank shows a device having a multiplicity of arches which act to maintain the tempromandibular joint in the proper position in an attempt to prevent bruxing. U.S. Pat. No. 6,675,804 to Pivovarov shows a device which maintains the tongue and mandibular and maxillary teeth in a position which seeks to eliminate the potential for bruxing. However, the device in Pivovarov encompasses both the mandibular and maxillary teeth, and is bulky and uncomfortable. U.S. Pat. No. 6,604,528 to Duncan shows a device that contains guide elements to position the device relative to the mouth and teeth and at the same time appears to prevent mating of the teeth when the mouth is closed and therefore gnashing or grinding of teeth during sleep. Yet, such devices heretofore shown in the art that are aimed to prevent bruxism or damage caused by bruxing fail to address or effectively aid in the treatment of snoring, OSA and GERD.

Hence, an object of the instant invention is the presentation of an effective intraoral appliance for treatment of sleep disorders including snoring, OSA, GERD or bruxing by moving the user's mandible forward with respect to the user's maxilla in order to open the airway in a manner which is comfortable, easily placeable, and without adverse effects. As shown above, prior devices have struggled to optimize the size and design parameters to facilitate the dual goals of comfort and effectiveness, but have woefully failed to achieve this goal. Thus, compliance is a predominant issue.

In addition, the prior art in this field includes the intraoral mandibular advancement device disclosed in the applicant's U.S. Pat. No. 7,730,891 (Lamberg). The Lamberg device addresses the shortcomings in the prior art discussed above by providing a curved protrusive element that distends from the maxillary appliance. The anterior aspect of this protrusive element contacts the posterior edge of a lingual spacer on the mandibular appliance and thereby causes mandibular advancement. The Lamberg device can be viewed as a type of “deprogrammer” to counteract clenching and bruxism. In particular, it relies on proprioceptive inhibition, which minimizes clenching when the anterior teeth are in contact. The literature in this field sometimes uses the terms “AMS” (anterior midpoint stop) or “APS” (anterior point stop) in discussing this instinct. However, it must be noted that proprioceptive inhibition reduces the contractibility of the mandibular elevator muscles, but does not eliminate it. The degree to which it inhibits muscle activity varies from person to person. Thus, some patients using the Lamberg device may still exhibit clenching and bruxing, which could overload the TMJ and result in damage to the joint and pain. Because clenching and grinding of the teeth is roughly twice as prevalent in patients with sleep disordered breathing (e.g., snoring or sleep apnea), there is a continued need for an appliance to manage both issues.

### Solution to the Problem

The present invention provides an intraoral mandibular advancement device that substantially eliminates or reduces disadvantages associated with prior devices in connection with sleep disorders. More specifically, the instant invention provides an intraoral mandibular advancement device for the treatment of sleep disorders, primarily snoring, and also including OSA, GERD or bruxism that urges the user's lower jaw forward via a protrusive element that pushes against the lingual surface of a mandibular appliance.

In particular, an intraoral mandibular advancement device is shown to treat problems associated with sleep disorders in a user having an obstructed oropharyngeal space, having a main body for attachment to the user's mouth and a protrusive element distending from the central portion of the maxillary appliance such that when worn by the user the protrusive element causes mandibular advancement sufficient to expand the oropharyngeal space and reduce the obstruction. Anterior movement of the mandible by the instant device serves to urge the attached tongue forward. For example, anterior movement of the mandible and tongue decreases the negative pressure in the esophagus and thereby reduces or eliminates GERD (when the same is caused thereby). Importantly, mandibular advancement, or protrusion, also creates more space behind the tongue and therefore increases the airway diameter in the oropharynx.

The protrusive element further serves to position the mandible in place to inhibit the tongue from moving posteriorly during sleep in the supine position. When the instant inventive device is inserted in the user's mouth, the protrusive element fits snugly against the lingual surface of the mandibular appliance.

Mandibular advancement effects the anatomy of the upper airway to allow increased air flow by, inter alia, (a) elevating the base of the tongue in resting position; (b) tensing the palatoglossus muscles and urging the soft palate (uvula) forward; (c) decompressing tissues around the pharynx and allowing the pharynx to expand; (d) stabilizing the lateral pharyngeal wall by applying tension to the pterygomandibular raphe, which is coupled to pharyngeal constrictors; and (e) splaying the tonsillar arches formed by the palatoglossus and the palatopharyngeal muscles, which leads to further stabilization of the lateral pharyngeal wall.

The present invention includes posterior stops on the posterior occlusal surfaces between the maxillary and mandibular appliances to maintain separation between the posterior upper and lower teeth. These posterior supports may reduce the proprioceptive inhibition of the anterior midpoint stop in the previous Lamberg device, but help to prevent overloading the TMJ. This is important if the patient either presents with temporomandibular disorder (TMD) or develops symptoms within the course of treatment.

Finally, the present invention includes an anterior stop on the occlusal aspect of the mandibular appliance that maintains an adjustable separation between the anterior upper and lower teeth. The posterior stops and the anterior stop provide three points of contact between the upper and lower teeth. It recognizes that when the jaw is protruded forward, the head of the mandibular condyles follows along the eminence of the maxilla, at a slope of approximately 30 degrees, which causes them to drop or separate from the maxillary teeth. This is known as the Christiansen effect. By reducing the height of the anterior stop as this occurs over the course of treatment, posterior and anterior contacts between the upper and lower appliances can be maintained throughout treatment.

### SUMMARY OF THE INVENTION

This invention provides an intraoral mandibular advancement device for treatment of sleep disorders that includes a maxillary appliance having a protrusive element distending from the anterior portion of the maxillary appliance, and a mandibular appliance having a polymeric shell with recesses to receive and removably engage the mandibular teeth. A lingual spacer extends posteriorly from the anterior portion of the mandibular appliance to contact the anterior aspect of the protrusive element and thereby cause mandibular advancement. An anterior stop extends upward from the occlusal surface of the anterior portion of the mandibular appliance to contact the anterior portion of the maxillary appliance and thereby maintain separation between the anterior teeth. Posterior stops on the occlusal surface of the posterior portions of the appliances maintain separation between the posterior teeth.

These and other advantages, features, and objects of the present invention will be more readily understood in view of the following detailed description and the drawings.

### BRIEF DESCRIPTION OF THE DRAWINGS

The present invention can be more readily understood in conjunction with the accompanying drawings, in which:

FIG. 1 is a top axonometric view of one embodiment of the maxillary appliance 1 in the present invention.

FIG. 2 is a bottom axonometric view of the maxillary appliance 1 in FIG. 1.

FIG. 3 is a bottom view of the maxillary appliance 1 in use on the maxillary teeth 20.

FIG. 4 is a top axonometric view of one embodiment of the mandibular appliance 10 in use on the mandibular teeth 30.

FIG. 5 is a bottom axonometric view of the mandibular appliance 10 corresponding to FIG. 4.

FIG. 6 is a detail vertical cross-sectional view of both appliances 1, 10 on the anterior teeth.

FIG. 7 is a vertical cross-sectional view similar to FIG. 6 showing the arc of opening and closing 95 as the mandible rotates about the condylar hinge axis 90.

FIG. 8 is a side view of the appliances 1, 10 in use on the teeth 20, 30 with the jaw partially open.

FIG. 9 is a side view corresponding to FIG. 8 with the jaw closed so that the posterior stops 16 contact the maxillary teeth 20 and the anterior stop 14 contacts the occlusal surface of the anterior portion of the maxillary appliance 1.

FIG. 10 is top axonometric view of another embodiment of the maxillary appliance 1 in which a thin polymeric shell with recesses removably engages the upper teeth 20. Posterior stops 8 extend downward from the maxillary appliance 1 to maintain a minimum spacing between the upper and lower teeth 20, 30.

FIG. 11 is a bottom axonometric view of the maxillary appliance 1 corresponding to FIG. 10.

### DETAILED DESCRIPTION OF THE INVENTION

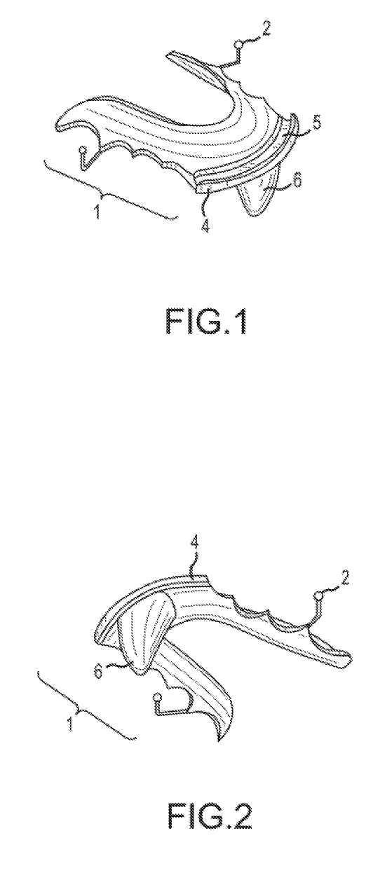

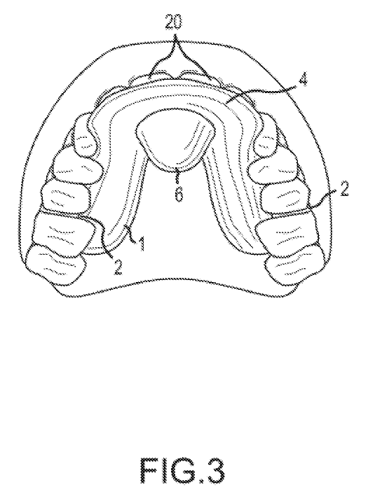

FIGS. 1 and 2 show two views of an embodiment of the maxillary appliance 1 in the present invention. This maxillary appliance 1 permits the user to voluntarily open and close his or her mouth to speak or drink a glass of water, while also allowing for natural movement during sleep. The maxillary appliance 1 has a main body that is customized to fit along the lingual surface of the user's maxillary teeth 20 and hard palate while leaving the soft palate and middle area of the palate uncovered during use. FIG. 3 is a bottom view of the maxillary appliance 1 in use on the maxillary teeth 20.

As shown in FIGS. 1 and 2, at the anterior end of maxillary appliance 1 is a retention arc 4 that is customized to fit a plurality of the user's anterior maxillary teeth for retention of the device in the user's mouth when worn during the user's sleep state. Retention arc 4 forms a trough 5 which, in this embodiment, conforms with the user's anterior maxillary teeth for positioning and serves to retain the maxillary appliance 1. The trough 5 formed by the retention arc 4 is, in the embodiment shown in FIGS. 1 and 2, less than 1.0 mm in depth so that the incisal edges of the user's maxillary incisors make contact with the appliance 1 for location, while the remainder of appliance 1 provides for retention. As shown in FIG. 3, the retention arc 4 covers only the maxillary central and lateral incisors, thus leaving the remaining maxillary teeth 20, including maxillary canine teeth, uncovered.

Extending from the lateral anterior portions of maxillary appliance 1 are clasps 2 for retention of the appliance 1 in the user's mouth. The clasps 2 are customized to cover the dental and exterior surfaces of the user's upper left and right molars, preferably the first molars. At the distal end of clasps 2 are clasp ends that fit adjacent to the exterior of the upper teeth gingival to the height of contour so as to engage the undercut in order to help retain the device in the user's mouth. The clasps 2 may comprise other retentive shapes without deviating from the letter and spirit of the instant invention.

Distending from the central portion of maxillary appliance 1 is a protrusive element 6. The protrusive element 6 is customized to conform with the lingual surface of the lingual spacer 12 on the mandibular appliance 10, as will be discussed below. This protrusive element 6 engages the lingual surface of the lingual spacer 12 so as to maintain a forward posture of the user's mandible relative to the maxilla. As the mandible moves forward, so does the attached tongue, thus maintaining the tongue in a forward posture. This causes mandibular advancement sufficient to expand the oropharyngeal space and reduce any obstruction therein, including the tongue. The length of the protrusive element 6 is preferably about 10.0 mm, yet the precise length will vary with the structure of the mouth of each user, and is customized to facilitate comfort and efficacy. Anterior movement of the mandible by the instant device serves to urge the tongue forward, thereby achieving the aforementioned goals, objects and features. For example, anterior movement of the mandible and tongue decreases the negative pressure in the esophagus and thereby reduces or eliminates GERD. Importantly, mandibular advancement, or protrusion, also creates more space behind the tongue and therefore increases the airway diameter in the oropharynx.

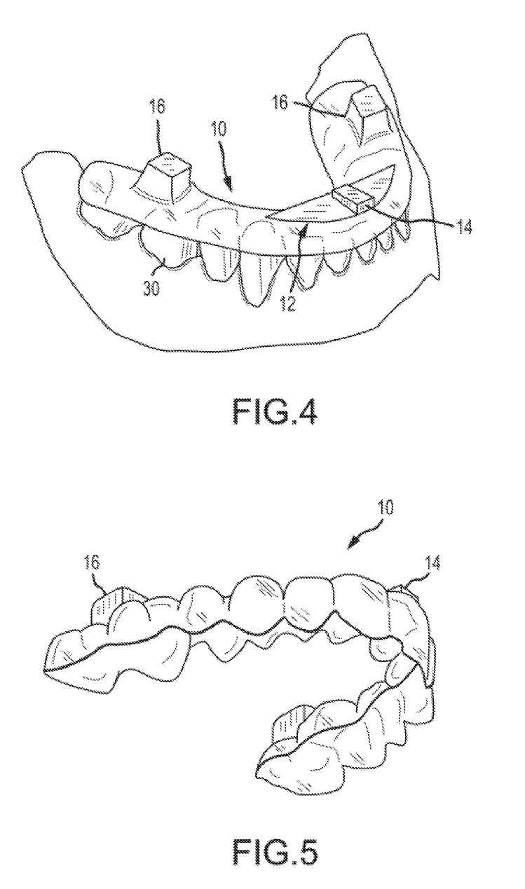

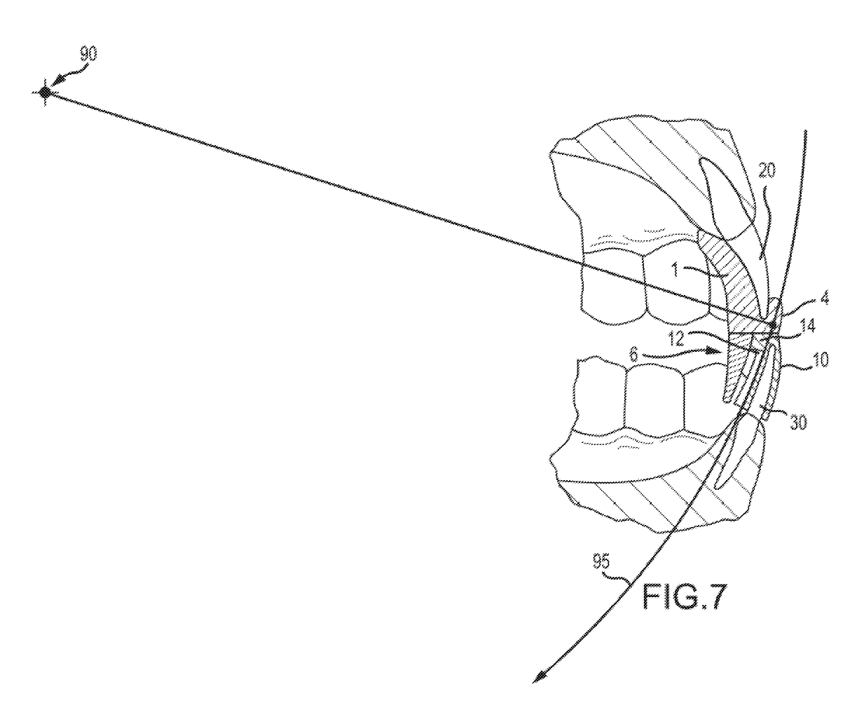

FIGS. 4 and 5 show an embodiment of the mandibular appliance 10 with a lingual spacer 12 extending posteriorly from the anterior mandibular teeth to create a contact for the protrusive element 6 of the maxillary appliance 1 to keep constant a desired degree of protrusion of the mandible at variable positions of mouth opening, irrespective of the shape or inclination of the lower teeth. As shown in FIG. 7, the protrusive element 6 has a leading edge consistent with the arc of opening and closing 95 of the TMJ. The lingual spacer 12 is essentially a “mate” for the protrusive element 6. The lingual spacer 12 can have an arcuate posterior surface and be mounted to a thin polymeric shell or aligner with recesses that receive at least some of the user's anterior mandibular teeth (e.g., the incisors) or all of the user's lower teeth, as shown in FIGS. 4 and 5. For example, the polymeric shell aligner 85 can be similar to those marketed by Align Technologies under the Invisalign trademark.

It should be noted that the protrusive element cannot reliably act against the lower incisors due to variability in their size, geometry and position. The lingual spacer 12 provides a fixed edge or surface for the protrusive element 6 to act against. Additionally, the thin shell of the mandibular appliance 10 helps to manage the risk of flaring or proclination of the lower teeth as a result of forces from the protrusive element 6.

Ideally in this embodiment, the anterior (or facial) aspect of the protrusive element 6 should be parallel to the “arc of opening and closing” 95, as illustrated in FIG. 7. This arc 95 has been well documented in the literature. Bonwill's Equilateral Triangle (1887), Monson's Spherical Theory (1932), and Weinberg's studies (1963) have described an arc that is defined by the motion of the incisal edges of the lower incisors as they rotate around the condylar hinge axis 90 of the mandible. This arc 95, as determined by a radius from the condylar hinge axis 90, has also been well illustrated in Posselt's envelope of mandibular movement. At any amount of protrusion, this arc 95 will typically be defined by a radius of about 100 mm or 4 inches (i.e., the distance from the hinge axis of the condyles to the lower incisors is generally constant irrespective of the protrusiveness).

The starting position of the lower incisors is typically between 32 mm and 50 mm below the horizontal plane of the condylar hinge axis 90 of the mandible. This geometric relationship will be fairly consistent because although the mandibular incisors will be initially about 4 mm lower than this, it should be noted that the mandibular condyles will also drop about the same amount vertically as they move down and forward along the posterior eminence of the maxilla (average inclination of 30 degrees-reference Gysi) during this protrusion. It is generally accepted to begin with a treatment position of about 60%-70% of the range of motion anterior to the most retruded position. Considering that the average range of motion is about 10-12 mm, therapy should be initiated in a position about 7 mm protruded from the maximum intercuspal position (MIP).

It is preferable that the lower jaw is advanced beginning at least 3.0-4.0 mm relative to the MIP and moved anteriorly as required to achieve the goals stated herein. This 3.0-4.0 mm shift represents approximately 50% of an individual's maximum active protrusive range. The optimal degree of offset will vary with each user and hence adjustability is provided.

It should be noted that this invention avoids the use of hardware that connects the two dental arches to enhance comfort and increase patient compliance, which remains one of the most significant challenges for the OSA patient. Although patients exhibit a range of vertical positions during sleep, few open more than 15 mm, which would be necessary to disengage the protrusive element 6 because it is about 10 mm long and sits on the 2 mm thick anterior stop platform on the occlusal surface of the maxillary appliance, and this must be added to 2-3 mm of normal overbite.

The mandibular appliance 10 can be fabricated by thermoforming a plastic aligner from canine to canine, or extended posteriorly to cover all the lower teeth. Preferably, the mandibular appliance 10 is complete aligner, as shown in FIGS. 4 and 5, having recesses formed to receive and removably engage all of the lower teeth, including the molars.

A lingual spacer 12 extends posteriorly from the lingual surface of the anterior portion of the mandibular appliance 10 (approximately 2 mm in vertical thickness) and extends horizontally in the lingual direction sufficiently to meet the leading edge of the protrusive element 6 so that it prevents the protrusive element 6 from engaging any other part of the lower teeth. For example, the lingual spacer 12 can extend posteriorly by about 1-4 mm depending on the inclination of the lower incisors and the shape of their cingulums. This will effect a generally constant amount of protrusion irrespective of the vertical opening of the mouth. Alternatively, the lingual spacer 12 could be a separately formed component that is bonded to the lingual surface of the mandibular appliance 10.

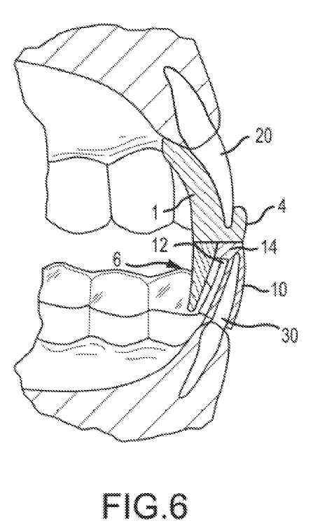

As shown in FIG. 4, the anterior portion of the mandibular appliance 10 can also be equipped with a generally horizontal, occlusal surface extending over the anterior mandibular teeth. For example, the horizontal occlusal surface can be continuation of the lingual spacer 12. An anterior stop 14 extends upward from the horizontal occlusal surface to maintain a predetermined minimum spacing or separation between the anterior portions of the maxillary and mandibular appliances 1, 10 as shown in FIG. 6. For example, the anterior stop 14 can be formed as a raised ridge of plastic on the occlusal surface of the anterior portion of the mandibular appliance 10. The minimum spacing between the anterior portions of the upper and lower appliances 1, 10 can be determined by adjusting the height of this ridge.

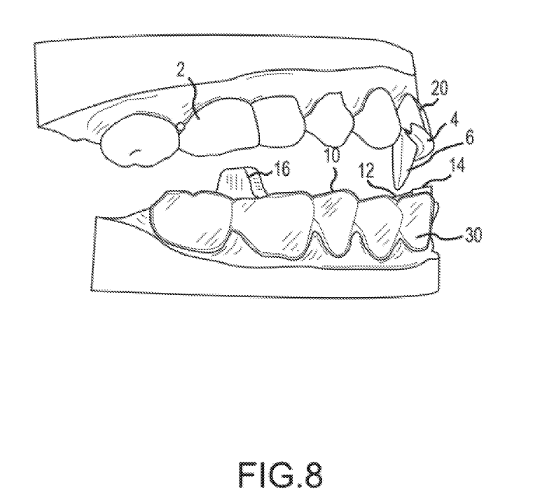

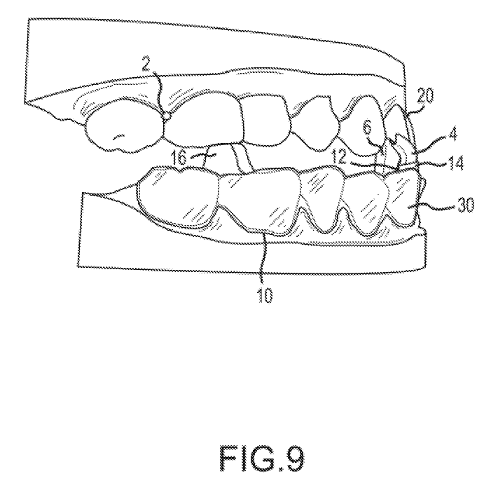

Posterior stops 16 extend upward from the occlusal surfaces of the posterior portions of the mandibular appliance 10 to maintain separation between the user's posterior maxillary and mandibular teeth 20, 30 as shown in FIGS. 8 and 9. The posterior stops 16 and the anterior stop 14 serve as a tripod of contacts to transfer forces between the upper and lower teeth 20, 30 and thereby avoid overloading the TMJ, which can cause damage to the joint and pain. FIG. 8 is a side view of the appliances 1, 10 in use on the teeth 20, 30 with the jaw partially open. FIG. 9 is a side view corresponding to FIG. 8 with the jaw closed so that the posterior stops 16 contact the maxillary teeth 20 and the anterior stop 14 contacts the occlusal surface of the anterior portion of the maxillary appliance 1.

Follow up and adjustment of the height of the anterior stop will be necessary over the course of treatment. As the jaw is protruded forward, the head of the mandibular condyles follows along the eminence of the maxilla, at a slope of approximately 30 degrees, thus separating the posterior teeth. This can be counteracted by progressively reducing the height of the anterior stop 14 (e.g., by grinding down the anterior stop 14) as the jaw positions forward to maintain the posterior contacts 16 in occlusion.

It is important to note that the present invention is not a deprogrammer, unlike the previous device disclosed in the Applicant's U.S. Pat. No. 7,730,891 (Lamberg). The device in the '891 patent was based on providing an anterior point stop to counteract clenching and bruxism, but specifically eliminated any contact between the posterior teeth. Instinct prevents clenching when the anterior teeth are in contact. Conventional wisdom holds that this instinct disappears if there is also contact between the posterior teeth. In contrast, the present invention provides posterior support to avoid overloading the TMJ. The present invention recognizes that the instinct to prevent clenching when the anterior teeth are in contact continues to a significant degree, and many patients will avoid clenching even with contact between the upper and lower posterior teeth.

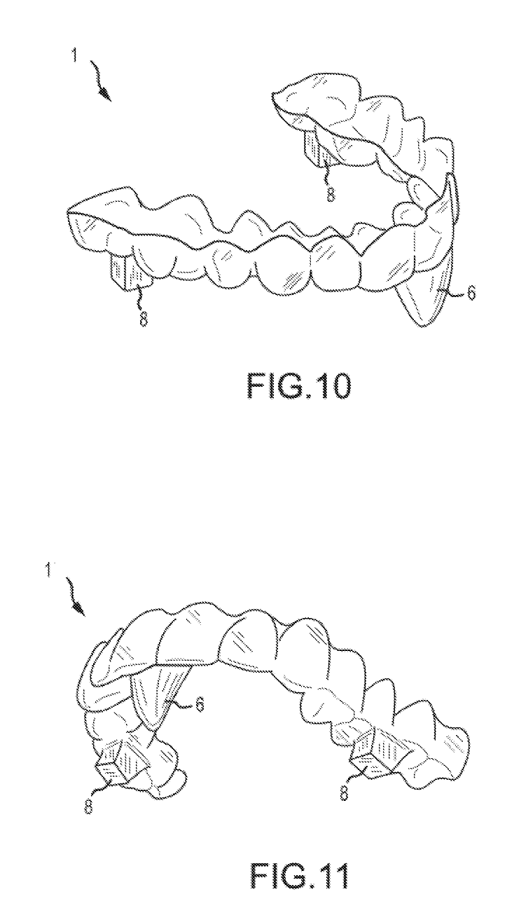

The embodiment of the upper appliance 1 shown in FIGS. 1-2 can be essentially the same as in U.S. patent application Ser. No. 7,730,891 (Lamberg). This embodiment doesn't need to cover the occlusal aspect of the posterior teeth, but rather is held in place by clasps 2, as previously discussed. FIGS. 10 and 11 shows an alternative embodiment of the maxillary appliance 1 in which a thin polymeric shell with recesses removably engages all of the upper teeth 20. In this embodiment, two posterior stops 8 extend downward from the occlusal surfaces of the maxillary appliance 1 to maintain a minimum spacing between the upper and lower teeth 20, 30. FIG. 11 is a bottom axonometric view of the maxillary appliance 1 corresponding to FIG. 10. This embodiment would enable the mandible appliance 10 to be reduced in size to cover only the anterior lower teeth. In that case, the posterior stops 8 would directly contact the lower posterior teeth. Alternatively posterior stops 8, 16 could be provided on both appliances 1, 10.

The above disclosure sets forth a number of embodiments of the present invention described in detail with respect to the accompanying drawings. Those skilled in this art will appreciate that various changes, modifications, other structural arrangements, and other embodiments could be practiced under the teachings of the present invention without departing from the scope of this invention as set forth in the following claims.

I claim:

1. An intraoral mandibular advancement device for treatment of sleep disorders in a user having an obstructed oropharyngeal space comprising:
a maxillary appliance to receive and removably engage a user's maxillary teeth and having a protrusive element distending from the anterior portion of the maxillary appliance;
a mandibular appliance having:
(a) a polymeric shell with recesses to receive and removably engage the user's mandibular teeth, said shell having an anterior portion, left and right posterior portions, and an occlusal surface;
(b) a lingual spacer extending posteriorly from the anterior portion of the mandibular appliance to contact the anterior aspect of the protrusive element and thereby cause mandibular advancement;
(c) an anterior stop extending upward from the occlusal surface of the anterior portion of the mandibular appliance to contact the anterior portion of the maxillary appliance and thereby maintain separation between the user's anterior teeth; and
(d) posterior stops on the occlusal surface of the posterior portions of the mandibular appliance maintaining separation between the user's posterior maxillary and mandibular teeth.

2. The intraoral mandibular advancement device of claim 1 wherein the anterior stop further comprises a ridge of polymeric material, and the separation between the user's anterior teeth is determined by changing the height of the ridge of polymeric material.

3. The intraoral mandibular advancement device of claim 1 wherein the height of the anterior stop is selected so that the anterior stop contacts the maxillary appliance when the posterior stops contact the maxillary teeth.

4. The intraoral mandibular advancement device of claim 1 wherein the posterior stops are made of polymeric material, and the separation between the user's posterior teeth is determined by changing the height of the posterior stops.

5. The intraoral mandibular advancement device of claim 1 wherein the lingual spacer further comprises an occlusal surface, and wherein the anterior stop extends upward from the occlusal surface of the lingual spacer.

6. An intraoral mandibular advancement device for treatment of sleep disorders in a user having an obstructed oropharyngeal space comprising:
a maxillary appliance having:
(a) a polymeric shell with recesses to receive and removably engage a user's maxillary teeth, said shell having an anterior portion, left and right posterior portions, and an occlusal surface;
(b) a protrusive element distending from the anterior portion of the maxillary appliance; and
(c) posterior stops on the occlusal surface of the posterior portions of the maxillary appliance maintaining separation between the user's posterior maxillary and mandibular teeth;
a mandibular appliance having:
(a) a polymeric shell with recesses to receive and removably engage the user's mandibular teeth, said shell having an anterior portion and an occlusal surface;
(b) a lingual spacer extending posteriorly from the anterior portion of the mandibular appliance to contact the anterior aspect of the protrusive element and thereby cause mandibular advancement; and
(c) an anterior stop extending upward from the occlusal surface of the anterior portion of the mandibular appliance to contact the occlusal surface of the anterior portion of the maxillary appliance and thereby maintain separation between the user's anterior teeth.

7. The intraoral mandibular advancement device of claim 6 wherein the anterior stop further comprises a ridge of polymeric material, and the separation between the user's anterior teeth is determined by changing the height of the ridge of polymeric material.

8. The intraoral mandibular advancement device of claim 6 wherein the height of the anterior stop is selected so that the anterior stop contacts the maxillary appliance when the posterior stops contact the maxillary teeth.

9. The intraoral mandibular advancement device of claim 6 wherein the posterior stops are made of polymeric material, and the separation between the user's posterior teeth is determined by changing the height of the posterior stops.

10. The intraoral mandibular advancement device of claim 1 wherein the lingual spacer further comprises an occlusal surface, and wherein the anterior stop extends upward from the occlusal surface of the lingual spacer.

11. An intraoral mandibular advancement device for treatment of sleep disorders in a user having an obstructed oropharyngeal space comprising:
a maxillary appliance to receive and removably engage a user's maxillary teeth and having a protrusive element distending from the anterior portion of the maxillary appliance;
a mandibular appliance having:
(a) a polymeric shell with recesses to receive and removably engage the user's mandibular teeth, said shell having an anterior portion, left and right posterior portions, and an occlusal surface;
(b) a lingual spacer having occlusal surface on the anterior portion of the mandibular appliance with a portion extending posteriorly to contact the anterior aspect of the protrusive element and thereby cause mandibular advancement;
(c) an anterior stop extending upward from the occlusal surface of the lingual spacer to contact the anterior portion of the maxillary appliance and thereby maintain separation between the user's anterior teeth; and
(d) posterior stops on the occlusal surface of the posterior portions of the mandibular appliance maintaining separation between the user's posterior maxillary and mandibular teeth.

12. The intraoral mandibular advancement device of claim 11 wherein the anterior stop further comprises a ridge of polymeric material, and the separation between the user's anterior teeth is determined by changing the height of the ridge of polymeric material.

13. The intraoral mandibular advancement device of claim 11 wherein the height of the anterior stop is selected so that the anterior stop contacts the maxillary appliance when the posterior stops contact the maxillary teeth.

14. The intraoral mandibular advancement device of claim 11 wherein the posterior stops are made of polymeric material, and the separation between the user's posterior teeth is determined by changing the height of the posterior stops.
